(19) United States
(12) Patent Application Publication
(10) Pub. No.: US 2015/0327751 A1
(43) Pub. Date: Nov. 19, 2015

(54) BENDABLE END-EFFECTOR

(71) Applicants: INDUSTRY-UNIVERSITY COOPERATION FOUNDATION HANYANG UNIVERSITY ERICA CAMPUS, Ansan-si, Gyeonggi-do (KR); KOH YOUNG TECHNOLOGY INC., Seoul (KR)

(72) Inventors: Hyun-Soo YOON, Ansan-si (KR); Byung-Ju YI, Bucheon-si (KR)

(21) Appl. No.: 14/347,772

(22) PCT Filed: Apr. 22, 2013
(86) PCT No.: PCT/KR2013/003377

§ 371 Date: Mar. 27, 2014

(30) Foreign Application Priority Data
Apr. 25, 2012 (KR) 10-2012-0043473
Apr. 26, 2012 (KR) 10-2012-0044130

(51) Int. Cl. A61B 1/005; A61B 17/22; A61B 1/00

(52) U.S. Cl. CPC A61B 1/0058; A61B 1/00085; A61B 17/22

(57) ABSTRACT

A bendable end-effector is described, used in endoscopic surgery which requires very large refraction angle, being capable of obtaining large internal space, as well as, transmitting safely a torque to a surgical instrument in curved state. The bendable end-effector includes a fixing block coupled at a driving unit, a bendable block coupled to the end of the fixing block and bending to a user's desired direction and angle, and a surgical instrument coupled to the end of the bendable block.

### TECHNICAL FIELD

Exemplary embodiments of the present invention relate to a bendable end-effector. More particularly, exemplary embodiments of the present invention relate to a bendable end-effector for an endoscopic surgery in otolaryngology such as paranasal sinuses.

### BACKGROUND ART

In general, an endoscopic surgery operates a surgery while watching an affected area of a patient through an image taken by an endoscope, in which surgical instruments and an endoscope camera are installed through a small hole, without making large incision.

Endoscopic surgery, started from laparoscopic surgery, has an advantage of a fast recovery time of surgical patients compared with open surgery since the incision is small and less bleeding.

Recently, endoscopic surgery system is technically developed enough to be used almost in all type of open surgery as well as it has being increasing in other field of medicine.

Meanwhile, otolaryngology, a branch of medical science, handles anatomy, physiology, and pathology of ear, nose, pharynx, and larynx, and applies an endoscope to various types of surgeries and treatments. But, there is a problem that endoscope and surgical instrument are difficult to access body parts, such as paranasal sinuses, and to be applied ENT endoscopic surgery due to a human body structure characteristic.

There is a problem that an end-effector are not made of flexible material since a surgical instrument should transmit force to operate, in which the surgical instrument used in ENT endoscopic surgery are biopsy, forceps, shaver, suction, and scissors.

Especially, end-effectors containing fine crusher for paranasal sinuses surgery, are rigid typed that are impossible to bend, or bent in a fixed angle like 5,15,45,and 60 degrees, may not reach to various areas in which tissues to be removed are positioned. Especially, it is a problem that there are inaccessible areas to reach, or have to operate punching treatment externally when a lesion exists in anterior and inferior region of maxillary sinus and interior of frontal sinus.

So, recently, it has been developed an end-effector including plurality of cylinders using coil springs.

But, such conventional end-effectors including plurality of cylinders using coil spring, has an limited angle capable to bend, maximum to 90 degree, also has a problem that it can't be used in a fine crusher for curved areas since a force to support the coil spring is restricted, is a force of restitution to original state is weak, and as well as, it is restrictive to obtain enough area in the interior when coupling the coil spring and the cylinder due to its structure characteristic.

### DISCLOSURE

### Technical Problem

Therefore, the aim of the present invention is to provide bendable end-effectors available in endoscopic surgery requiring very large curved angle, support strong force, and as well as, to provide stable torque in curved state.

### Technical Solution

In one embodiment of the present invention, a bendable end-effect includes a fixing block coupled to a driving unit, a bendable block coupled to the end of the fixing block and capable of bending to a desired direction and angle that a user wishes, and a surgical instrument coupled to the end of the bendable block.

In one embodiment, the bendable block includes a first plurality of cylinders aligned in an interval, a second plurality of cylinders alternately aligned between any of two of the first plurality of cylinders in which the first plurality of cylinders are adjacent to each other, a first pair of band-type springs coupling each of the first cylinders to each of the second cylinders such that each of the second cylinders are bent to transverse or longitudinal direction based on each of the first plurality of cylinders, and a second pair of band-type springs coupling each of the first cylinders to each of the second cylinders such that each of the first plurality of cylinders are bent perpendicular to the second plurality of cylinders based on each of the second plurality of cylinders.

Herein, the first and second pairs of band-type springs may couple the first and second plurality of cylinders, such that a center line of the first and second plurality of cylinders is parallel to widths direction of the first and second pairs of band-type springs.

In one embodiments, the first plurality of cylinders may include a body with a hollow shape, a first pair of coupling slots formed in a side of the body such that an end portion of another side of the first pair of band-type springs are inserted and coupled to the first coupling slot, and a second pair of coupling slots formed on another side of the body such that an end portion of a side of the second pair of band-type springs are inserted and coupled to the second coupling slot.

Herein, the first plurality of cylinders may further include a first wire through-hole passing through the body such that the wire is disposed both side of the first pair of coupling slots, and a second wire through-hole passing through the body such that the wire is disposed both side of the second pair of coupling slots.

In one embodiment, the second cylinder may include a body with a hollow shape, a first pair of coupling slots formed on a side of the body such that an end portion of one side of the first pair of band-type springs is inserted and coupled to the first pair of coupling slots, and a second pair of coupling slots formed on another side of the body such that an end portion of another side of the second pair of band-type springs is inserted and coupled to the second pair of coupling slots.

Herein, the second cylinder may further include a first wire through-hole formed on both side of the first pair of coupling slots to pass through the body, and a second wire through-hole formed on both side of the second pair of slots to pass through the body.

In one embodiment of the present invention, the bendable end-effector may further include a power transmission coupled to the driving unit and the surgical instrument such that torque of the driving unit may be transmitted to the surgical instrument.

Herein, the surgical instrument may be a shaver.

In one embodiment, the power transmission includes a first coupling block coupled to the driving unit, a wire coupled to the first coupling block to be flexible to a direction and angle of the bendable block, and a second coupling block coupled to the wire such that the wire are coupled to the surgical instrument.

Herein, the wire may be a SUS wire.

Meanwhile, it is preferable to couple the second coupling block and the surgical instrument in “T”-shaped form to obtain enough space for suction space.

### Advantageous Effects

Thus, according to an embodiment of the present invention, the bendable end-effector may be used in endoscopic surgery which requires various and very large refraction angle since the first and second band-type springs are coupled to the first and second cylinders such that it is possible to bend in longitudinal or transverse direction over 180 degrees without changing tip. Therefore, endoscopic surgery range may be extended.

Also, it is possible to obtain the entire internal space of the first and second cylinders since the first and second band-type springs are not protruded to the interior of the first and second cylinders.

Therefore, surgical instrument may be applied more widely as the end-effector may be coupled to a camera, a biopsy, and a crusher, as well as, a suction that is not coupled without obtaining the entire internal space, since the entire internal space of the first and second cylinders is obtained.

In addition, the bendable end-effector according to the present invention, the second band-type springs may support adjacent cylinders without being bent when the cylinders are bent in longitudinal or transverse direction through the first band-type springs. Meanwhile, the first band-type springs may support adjacent cylinders without being bent when the cylinders are bent in a longitudinal or transverse direction through the second band-type springs.

So, the bendable end-effector according to the present invention may support more power than conventional end-effector in which a coil spring are used to couple plurality of cylinders aligned in a line, as well as, has better restoring force to an original state.

In addition, the bendable end-effector according to the present invention, a lesion located in a various angle would be removed easily and safely during the endoscopic surgery since the bendable block may transmit safely torque of the driving unit to the surgical instrument in a curved state through a power transmission.

### DESCRIPTION OF DRAWINGS

FIG.1 is a diagram showing an end-effector according to an embodiment of the present invention;

FIG. 2 is a diagram showing an end- effector according to another embodiment of the present invention;

FIG. 3 is a diagram showing an end- effector according to another embodiment of the present invention;

FIG. 4 is a detailed view of A of FIG.1;

FIG. 5 is a diagram showing a coupling state of a surgical instrument and a power transmission;

FIG. 6 is a diagram showing a coupling state of the surgical instrument and a second coupling block;

FIG. 7 is a diagram explaining a first cylinder according to an embodiment of the present invention;

FIG. 8 is a detailed view of B of FIG. 7;

FIG. 9 is a detailed view of C of FIG. 7;

FIG. 10 is a diagram explaining a second cylinder according to an embodiment of the present invention;

FIG. 11 is a detailed view of D of FIG. 10;

FIG. 12 is a detailed view of E of FIG. 10.

### MODE FOR INVENTION

The present invention is described more fully hereinafter with reference to the accompanying drawings, in which example embodiments of the present invention are shown.

The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that, although the terms first, second, third etc. may be used herein to describe various elements, components, regions, layers and/or sections, these elements, components, and/or sections should not be limited by these terms. These terms are only used to distinguish one element, component, region, layer or section from another region, layer or section. Thus, a first element, component, or section discussed below could be termed a second element, component, or section without departing from the teachings of the present invention.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

For convenience, same numerals are used for identical or similar elements of an apparatus of cutting a tempered substrate and the conventional one.

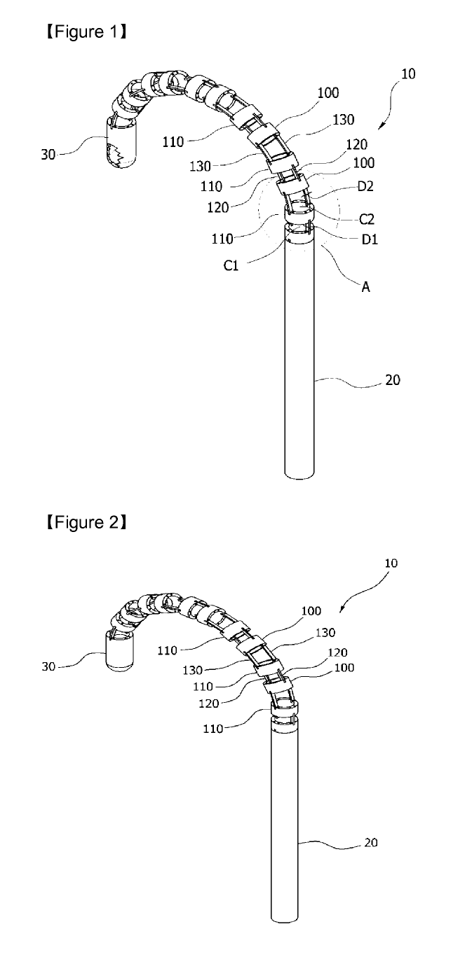

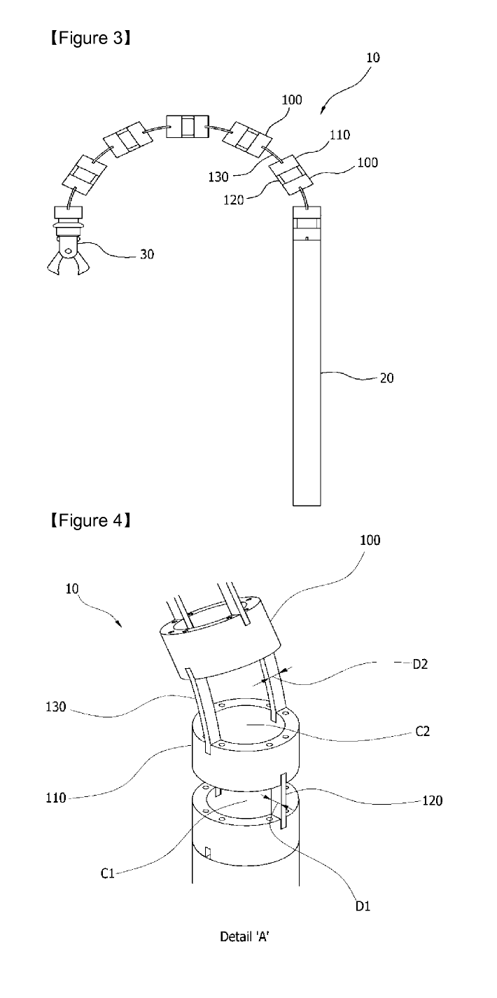

Hereinafter, with reference to the drawings, preferred embodiments of the present invention will be described in detail. FIG. 1 is a diagram showing an end-effector according to the present invention, FIGS. 2 and 3 are diagrams showing other end-effector of the present invention, and FIG. 4 is a detailed view of A of FIG. 1.

Referring to FIGS. 1 to 4, a bendable end-effector includes a fixing block 20 and a bendable block 10.

The fixing block 20 has hollow shape, and disposed on a driving unit (not shown) in which an operating tool (not shown) is mounted.

The bendable block 10 is coupled to an end of the fixing block 20, such that a user can bend to a desired direction and angle.

The bendable block 10 includes a first cylinders 100, a second cylinders 110, a first band-type springs 120, and a second band-type 130 springs.

Herein, one of the first and second cylinders located on both end of one side is coupled to the fixing part 140, and a surgical instrument described later is coupled to another cylinder located on both end of one side. The first cylinders 100 are circular ring shaped with a center hole. The plurality of second cylinders 110 are disposed between the first cylinders 100 which are aligned in line with an interval. In other words, the first cylinders and the second cylinders are aligned alternatively with the first cylinders.

The first pair of band-type springs making a couple connects each of the second cylinders 110 to the first cylinders 100 such that the second cylinders 110 are bent based on the first cylinders 100. For example, the first pair of band-type springs 120 couples each of the first cylinders 100 and each of the second cylinders 110 based on the first cylinders 100 such that the second cylinders 110 are bent only to a transverse direction. Alternatively, the first pair of band-type springs 120 couples each of the first cylinders 100 and each of the second cylinders 110 based on the first cylinders 100 such that the second cylinders 110 are bent only to a longitudinal direction.

The second pair of band-type springs 130 making a couple connects each of the second plurality of cylinders 110 to each of the first plurality of cylinders 100 based on each of the second cylinders 110, such that the first cylinders 100 are bent perpendicular to a bending direction of each of the second cylinders 110. For example, in case that the first pair of band-type springs 120 couples each of the first cylinders 100 to each of the second cylinders 110 such that each of the second cylinders 110 is bent only in transverse direction based on each of the first cylinders 100, the second pair of second band-type springs 130 couples each of the second cylinders 110 to each of the first cylinders 100 such that each of the first cylinders 100 is bent in a longitudinal direction based on each of the first cylinders 100. Alternatively, in case that the first pair of band-type springs 120 couples each of the first cylinders 100 to each of the second cylinders 110 such that each of the second cylinders 110 is bent only in longitudinal direction based on each of the first cylinders 100, the second pair of band-type springs 130 couples each of the second cylinders 110 to each of the first cylinders 100 such that each of the first cylinders 100 is bent in a transverse direction based on each of the second cylinders 110.

In this way, the bendable block 10 supports the adjacent cylinders 100 and 110 without bending the second band-type springs 130 when the cylinders 100 and 110 are bent to a transverse or longitudinal direction by the first band-type springs 120. Meanwhile, the first pair of band-type springs 120 are not bent and support the adjacent cylinders 100 110 when the cylinders 100 120 are bent to a transverse or longitudinal direction by the second band-type springs 130. Therefore, a user can easily bend the bendable block to a desired direction and angle. It has an advantage that may support more force than conventional end-effector which uses a coil spring to couple a plurality of cylinders, as well as, has better restoring force to an original state.

Therefore, it has an advantage in extending the scope of surgical instruments since it receives small restriction on mounting surgical instruments according to driving force compared with the conventional end-effector using coil spring.

In addition, the bendable end-effector 10 according to the present invention has an advantage that may be applied in endoscopic surgery which requires very large refraction angle, such as paranasal sinuses, since the first and second cylinders 100 and 110 are coupled to the first and second band-type springs 120 and 130 over 180 degree.

Meanwhile, a pair of the first and second band-type springs 120 and 130 making a couple connect each of the first and second cylinders 10 and 110 such that the direction of the widths D1 and D2 is parallel to a center line C1 and C2 of the pair of the first and second cylinders 100 and 110.

Alternatively, the pair of the first and second band-type springs 120 and 130 making a couple connect the first and second cylinders 100 and 110 such that the direction of the widths (not shown) D1 and D2 are perpendicular to the center line C1 and C2 of the first and second cylinders 100 and 110.

Meanwhile, the end-effector according to the present invention may further include a surgical instrument.

The surgical instrument 30 is installed on an end portion of the bendable block 10. For example, the surgical instrument 30 may be installed on one of the first and second cylinders 100 and 110 which one is located on both end of one side. For example, a fine crusher may be used for the surgical instrument 30 as showed in FIG.1, a camera as showed in FIG. 2, or a biopsy as showed in FIG. 4. In addition, a suction (not shown) may be used for the surgical instrument 30.

Meanwhile, the driving unit (not shown) of the end-effector according to the present invention, may further include a power transmission 40 which transmits torque to the surgical instrument 30.

The power transmission 40 may be applied to an end-effector of the present invention, showed in FIG. 1, torque of the driving unit is transferred to the surgical instrument 30 when a fine crusher is used as a surgical instrument.

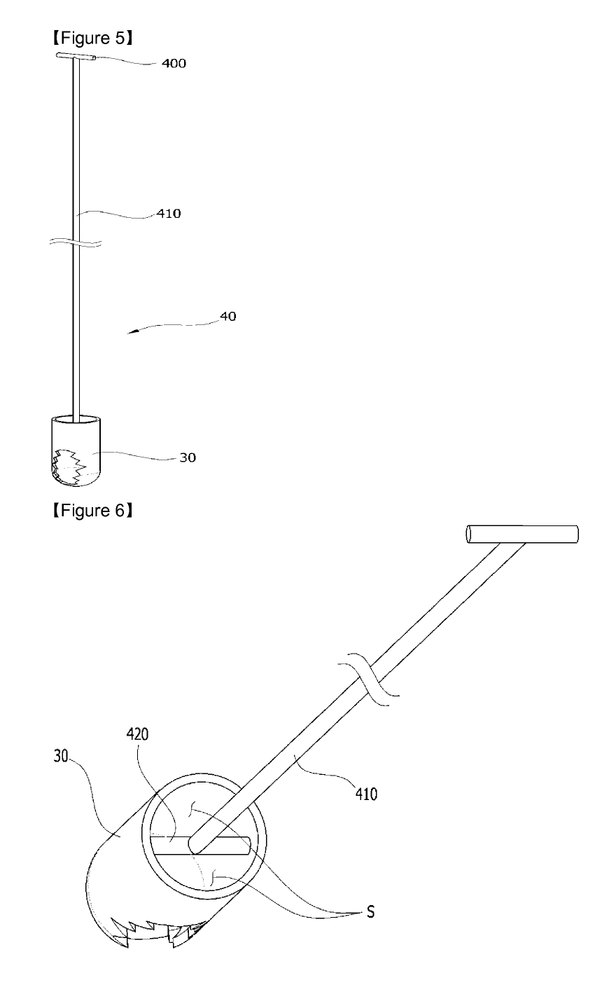

Referring to FIGS. 5 and 6 4, the power transmission 40 is described in detail.

FIG. 5 is a diagram showing a coupling state of a surgical instrument and a power transmission tool and FIG. 6 is a diagram showing a coupling state of the surgical instrument and a second coupling block.

Referring to FIGS. 5 and 6, the power transmission 40 passes through the fixing block 20 and the bendable block, and couples the driving unit and the surgical instrument 30 such that torque of the driving unit is transmitted to the surgical instrument 30 such as shaver. Herein, the power transmission tool 40 may be bent flexibly to a direction and angle of the bendable block 10. For example, the power transmission 40 includes a first coupling block 400, a wire 410, and a second coupling block 420. The first coupling block is connected to the driving unit. The wire 410 is coupled to the first coupling block and is bent flexibly to a bent direction and angle of the bendable block 10. The second coupling block 420 is coupled to the wire 410 and couples the wire and the surgical instrument 20. Herein, it is preferable to couple the first and second coupling blocks 400 and 420 and the wire in “T” shaped form. Specially, to obtain suction space(S) between the second coupling block 420 and the wire 410, the wire 410 may be coupled in “T” shaped to the surgical instrument 30. It is preferable to use a SUS wire as an example of the wire 410.

The bendable end-effector according to the present invention, it is possible to transmit safely the torque of the driving unit to the surgical instrument 30, such as shaver, in curved state since the driving unit transmits the torque to the surgical instrument 30 through the flexible wire 410, and there is an advantage that a lesion located in a various angle is removed safely.

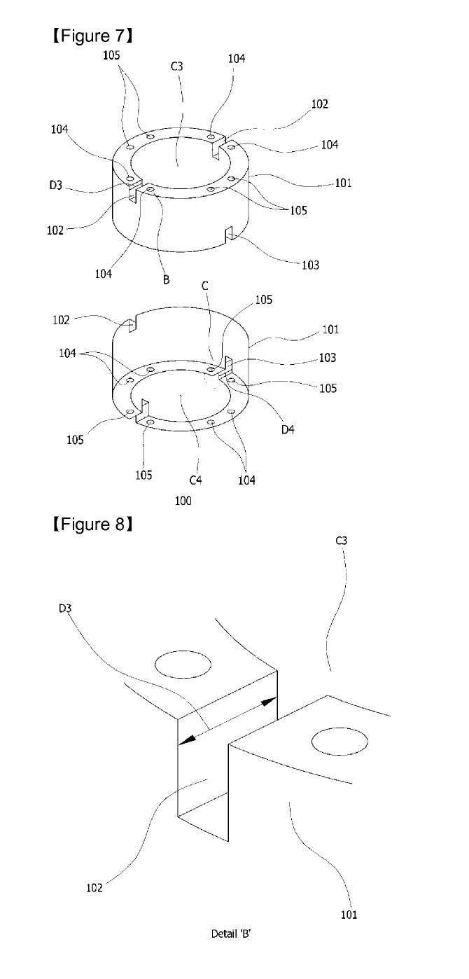

Next, a configuration of the first cylinder is described in detail according to FIGS. 7 to 9.

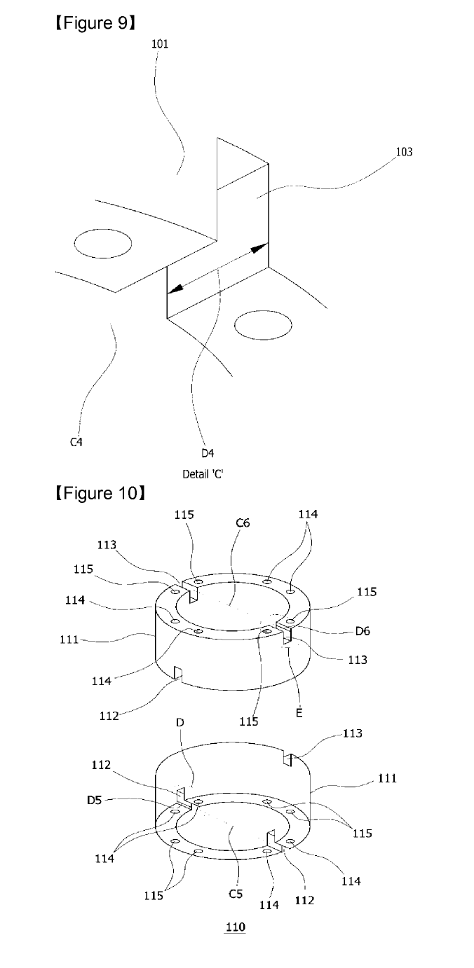

FIG. 7 is a diagram describing the first cylinder according to an embodiment of the present invention, FIG. 8 is a detailed view of B of FIG. 7, and FIG. 9 is a detailed view of C of FIG. 7.

Referring to FIGS. 7 to 9, the first cylinder 100 of the present invention includes a body 101, a first pair of coupling slots 101, and a second pair of coupling slots 103.

The body 101 has a hollow shape and cut in predetermined length to be circular form.

The first pair of coupling slots 102 is formed on a side of the body 101 such that an end portion of another side of the first band-type springs 120 is inserted and coupled to the first pair of coupling slots 102. For example, the first pair of coupling slots 102 is disposed facing (horizontally) the one side of the body such that the end portion of another side of first band-type springs 120 (Refer FIG. 1) is inserted and coupled to the first pair of coupling slots 102. Meanwhile, the first pair of coupling slots 102 is formed on a side of the body 100 such that the direction of the widths D3 is parallel to a center line C3 of the first cylinder 100. Alternatively, not shown in the figure, the first pair of coupling slots 102 may be formed on a side of the body 100 such that the direction of widths D3 is perpendicular to the center line C3 of the first cylinder 100.

The second pair of coupling slots 103 is formed on another side of the body 100 such that an end portion of one side of the second pair of band-type springs 130 (Refer to FIG. 1) is inserted and coupled to the second pair of coupling slots 103. In one embodiment, the second pair of coupling slots 103 is disposed facing (horizontally) another side of the body 101. Herein, the second pair of coupling slots 103 is formed on another side of the body 101 to be perpendicular to the first pair of coupling slots 102. Meanwhile, the second pair of coupling slots is formed on another side of the body 100 such that a direction of widths D4 is parallel to a center line C4 of the first cylinder 100. Alternatively, not shown in the figure, the second pair of coupling slots is formed on another side of the body 101 such that the direction of the widths D4 is perpendicular to the center line C4 of the first cylinder 100.

Therefore, the first cylinder 100 includes each pair of first and second coupling slots 102 and 103, in which the end of each of the first and second band-type springs are inserted and coupled, and so, it is possible to obtain the entire interior space of the first cylinder 100 since the first and second band-type springs 120 and 130 are not protruded to the interior of the first cylinder 100.

Meanwhile, the first cylinder 100 further includes a first and second wire through holes. The first and second wire through-holes 104 105 are holes in which the wires (not shown) pass through, and the wires passing through the holes may bend the bendable block 10 to a transverse or longitudinal direction.

Herein, the first wire through-hole 104 is formed on both side of the first coupling slots penetrating the body 101. Also, the second wire through-hole is formed on both sides of the second coupling slots 103 penetrating the body 101.

Next, referring to FIGS. 10 to 12, a configuration of the second cylinder is described in detail.

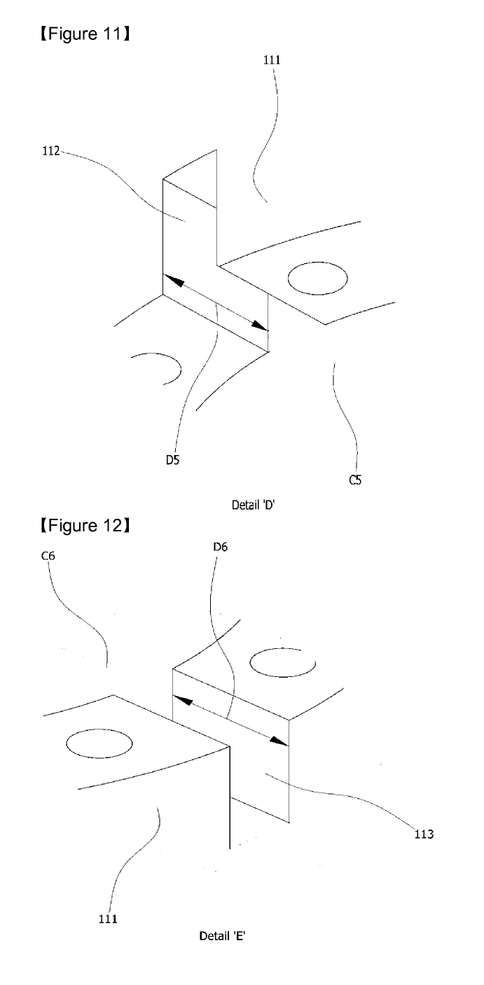

FIG. 10 is a diagram showing the second cylinder according an embodiment of the present invention, FIG. 11 is a detailed view of D of FIG.10 and FIG. 12 is a detailed view of E of FIG. 10.

Referring to FIGS. 10 to 12, the second cylinder 110 of the present invention includes a body 111, a first pair of coupling slots 112, and a second pair of coupling slots 113.

The body 111 has a hollow shape and predetermined length.

The first pair of coupling slots 112 is formed on another side of the body 113 such that an end portion of one side of the first pair of band-type springs (Refer to FIG. 1) is inserted and coupled to the first pair of coupling slots 112. For an instance, the first pair of coupling slots 112 is disposed facing (horizontally) another side of the body 111 such that the first band-type springs 120 is inserted and coupled to the first pair of coupling slots 112. In other words, the first pair of coupling slots is formed on another side of the body 111 such that the first pair of the coupling slots 112 corresponds to the first pair of coupling slots 102. Meanwhile, the first pair of coupling slots 112 is formed on another side of the body 110 such that a direction of the widths D5 is parallel to a center line C5 of the second cylinder 110. Alternatively, the first pair of coupling slots 112, not shown in the figure, may be formed on another side of the body 110 such that the direction of the widths D5 is perpendicular to the center line C5 of the second cylinder 110.

The second pair of coupling slots 113 is formed on a side of the body 110 such that an end portion of another side of the second pair of band-type springs (Refer to FIG. 1) is inserted and coupled to the second pair of coupling slots 113. For an instance, the second pair of coupling slots is formed facing (horizontally) the one side of the body 110. In other words, the second pair of coupling slots is formed on a side of the body 110 such that the second pair of coupling slots 113 corresponds to the second pair of coupling slot 103 of the first cylinder 100. Herein, the second pair of coupling slots 113 is formed on a side of the body 110 to be perpendicular to the first pair of coupling slots 103. Meanwhile, the second pair of coupling slots 113 is formed on a side of the body 110 such that the direction of widths D6 is parallel to a center line C6 of the second pair of cylinders 110. Alternatively, the second pair of coupling slots 113, not shown in the figure, may be formed on a side of the body 110 such that the direction of a widths D6 is perpendicular to the center line C6 of the second pair of cylinders.

Thus, the second cylinder 110 includes a pair of first and second coupling slots 112 and 113, in which the end of each of the first and second band-type springs 120 130 are inserted and coupled, such that it is possible to obtain the entire internal space of the second cylinder 110 since the end of the first and second band-type spring 120 and 130 are not protruded to the interior of the second cylinder 110.

Meanwhile, the second cylinder 110 further includes a first and second wire through holes 114 and 115. Also, the first and second wire through-holes 114 and 115 are holes in which the wires (not shown) pass through, and the wires passing through the holes may bend the bendable block 10 to a transverse or longitudinal direction like the first and second wire through holes 104 and 105.

Herein, the first wire through-hole 114 is formed on both side of the first coupling slots 112 penetrating the body 110. Also, the second wire through-hole 115 is formed on both side of the second coupling slots 103 penetrating the body 110.

Thus, according to an embodiment of the present invention, the bendable end-effector 10, may obtain the entire interior space of the first and second cylinders 100 and 110 since the first and second band-type springs 120 and 130 which connects the first and second cylinders 100 and 110 to each other are not protruded to the interior of the first and second cylinders 100 and 110.

Thus, the bendable end-effector 10 according to the present invention, as described above, surgical instrument may be applied more widely since it is possible to obtain the entire internal space of the first and second cylinders, surgical instrument can be applied to more widely since the end-effector can be coupled to a camera, a biopsy, and a shaver, as well as, a suction which can't be coupled without obtaining the entire internal space.

Also, the bendable end-effector 10 according to the present invention, has an advantage that endoscopic surgery range will be extended since the end-effector is bent in longitudinal or transverse direction over 180 degrees by coupling the first and second cylinders 100 and 110 through the first and second band-type springs 120 and 130.

It will be apparent to those skilled in the art that various modifications and variation can be made in the present invention without departing from the spirit or scope of the invention. Thus, it is intended that the present invention cover the modifications and is variations of this invention provided they come within the scope of the appended claims and their equivalents.

1. A bendable end-effector comprising:
a fixing block installed on a driving unit;
a bendable block coupled to an end of the fixing block and bending to a desired direction and angle that user wishes ; and
a surgical instrument coupled to the end of the bendable block.

2. The bendable end-effector of claim 1, wherein the bendable block comprises:
a first plurality of cylinders aligned in a line with an interval;
a second plurality of cylinders alternately aligned between any of two of the first plurality of cylinders, wherein the first plurality of cylinders are adjacent to each other;
a first pair of band-type springs coupling each of the first cylinders to each of the second cylinders to allow each of the second cylinders to be bent in transverse or longitudinal direction based on each of the first cylinders; and
a second pair of band-type springs coupling each of the second cylinders to each of the first cylinders to allow each of the first cylinders to be bent perpendicular to the second cylinders based on each of the second cylinders direction.

3. The bendable end-effector of claim 2, wherein the first and second band-type springs couple the first and second cylinders such that a direction of widths of the first and second band-type springs is parallel to a center line of the first and second cylinders.

4. The bendable end-effector of claim 2, wherein the first and second band-type springs couple the first and second cylinders to each other such that a direction of the widths of the first and second band-type springs is perpendicular to a center line of the first and second cylinders.

5. The bendable end-effector of claim 2, wherein the first cylinder comprises:
a body having hollow shape;
a first pair of coupling slots formed on a side of the body such that another end portion of the first pair of band-type springs are inserted and coupled to the first pair of coupling slots; and
a second pair of coupling slots formed on another end portion of the body such that an end portion of the second pair of band-type springs are inserted to the second pair of coupling slots

6. The bendable end-effector of claim 5, wherein the first cylinder comprises:
a first wire through-hole formed through the body such that the first wire through-hole is positioned on both side of the first pair of coupling; and
a second wire through-hole formed through the body such that the second wire through- hole is positioned on both side of the first pair of coupling.

7. The bendable end-effector of claim 2, wherein the second cylinder comprises:
a body having hollow shape;
a first pair of coupling slots formed on another side of the body such that an end portion of the first pair of band-type springs are inserted and coupled to the first pair of coupling slots; and
a second pair of coupling slots formed on a side of the body such that another end portion of the second pair of band-type springs are inserted and coupled to the second pair of coupling slots.

8. The bendable end-effector of claim 7, wherein the second cylinder comprises:
a first wire through hole formed through the body such that the first wire through hole is positioned on both side of the first pair of coupling slots; and
a second wire through hole formed through the body such that the second wire through hole is positioned on both side of the second pair of coupling slots.

9. The bendable end-effector of claim 1, further comprising:
is a power transmission coupled to the driving unit and the surgical instrument, wherein the power transmission is capable of being bent flexibly along a direction and angle of the bendable block to transmit a torque of the driving unit to the surgical instrument.

10. The bendable end-effector of claim 9, wherein the power transmission tool comprises:
a first coupling block coupled to the driving unit;
a wire coupled to the driving unit, wherein the wire is capable of being bent flexibly along a direction and angle of the bendable block; and
a second coupling block coupled to the wire to connect the wire and the surgical instrument.

11. The bendable end-effector of claim 10, wherein the wire comprises a SUS wire.

12. The bendable end-effector of claim 10, wherein the second coupling block is coupled to the wire in “T”-shaped form to connect the wire to the surgical instrument for obtain suction space between the second coupling block and the surgical instrument.

13. The bendable end-effector of claim 10, wherein the surgical instrument comprises a fine crusher.
